(19) United States
(12) Patent Application Publication
(10) Pub. No.: US 2007/0098687 A1
(43) Pub. Date: May 3, 2007

(54) Pharmaceutical composition containing interferon for the treatment of hpv infections

(76) Inventor: Giulio Tarro, Napoli (IT)

Correspondence Address: YOUNG & THOMPSON, 745 SOUTH 23RD STREET, 2ND FLOOR, ARLINGTON, VA, 22202, US

(21) Appl. No.: 10/553,387

(22) PCT Filed: Apr. 6, 2004
(86) PCT No.: PCT/EP04/03685

§ 371 Date: Jun. 19, 2006

(30) Foreign Application Priority Data
Apr. 18, 2003 (IT) MI2003A000826

(51) Int. Cl. A61K 38/21

(52) U.S. Cl. 424/85.4

(57) ABSTRACT

Disclosed is a liquid pharmaceutical composition containing interferon for peroral administration, and the use thereof in the treatment of infections caused by human papilloma virus (HPV).

The present invention provides a liquid pharmaceutical composition for peroral administration containing interferon, useful for the treatment of infections caused by human papilloma virus (HPV).

### BACKGROUND OF THE INVENTION

Some 120 different types of papilloma viruses have been so far identified, which infect humans provoking an epithelial or fibroepithelial proliferation of the skin or mucosae and consequently warts and condyloma lesions. Genital infections, which in some cases give rise to neoplasias such as squamous carcinoma and uterus cervix adenocarcinoma, are among the most diffused HPV-related diseases. In a multicentric study carried out in different countries, the HPV types 16 and 18 were found to be responsible for 70% of the uterus squamous carcinomas, while different HPV types were associated with uterus epithelial tumors.

Papilloma virus infections are persistent and hard to eradicate therefore requiring a repeated therapeutic treatment and in-time monitoring of patients for relapses or development of pre-cancerous lesions.

Since the virus accumulates in the lesion sites, the choice therapeutic treatment should be aimed at controlling the diffusion of infection by removing warts or visible pre-cancerous lesions, by topical therapy, laser therapy, criotherapy or surgery. Such treatments, however, do not ensure complete elimination of the virus, which thus can start a new infective process.

### DESCRIPTION OF THE INVENTION

It has been found that HPV-infected individuals can be treated with a liquid pharmaceutical composition containing low doses of interferon, to be administered by peroral route. The treatment according to the invention proved particularly effective, allowing complete elimination of the virus with only few applications.

The invention therefore provides the use of interferon for the preparation of a liquid pharmaceutical composition suitable for peroral administration, for use in the treatment of HPV infections. As used herein, peroral administration means contacting the interferon composition with the oral or pharyngeal mucosa for a time sufficient to allow adsorption/absorption of the active substance and the stimulation of immunocells and cytokine secretion at local and peripheral level through the lympathic system and blood stream. Suitable pharmaceutical forms include solutions, suspensions, dispersions, syrups or other liquid preparations containing pharmaceutically acceptable excipients. Water solutions containing buffering agents, salts and optionally stabilizers, adsorption/absorption enhancers, sweeteners, flavourings and cosolvents, are preferred.

The amount of interferon in the composition can range from 100 to 500 International Units (IU)/ml, preferably 150 IU/ml. According to a preferred posology scheme, from 0.5 to 10 ml, preferably 1 ml doses were administered one or two times a day, allowing a daily intake of from 150 to 15000 IU interferon. The daily amount can be modified depending on the severity of the disease, the general conditions of the patient, and other variable parameters. A synthetic interferon (e.g. recombinant) can be used, but the natural molecule, which contains different isoforms (α, β, γ) and subtypes, is preferred. Human natural interferon, preferably the a form, can be produced and purified from peripheral blood leukocytes or lymphoblastoid cells, according to known procedures, as described in U.S. Pat. No. 4,732,683; Cantell K. And Hirvonen S., Texas Reports on Biology and Medicine, vol. 35, p. 138, 1977; Zoon K. C. et al., Science 207, p. 527, 1980.

The peroral administration allows an immediate availability of interferon as well as the complete assumption of the desired amount thereof; in addition, it increases the patient's compliance and, of particular importance industrially, reduces the costs for the preparation, storage and distribution of the product, compared to currently used interferon formulations.

The treatment according to the invention was tested in a clinical study involving women positively diagnosed for HPV infection, to whom a solution of human interferon-α (150 IU per dosage unit) was administered for a period of 90 days or more. The treatment resulted effective in gradually reducing the initial quantity of virus up to its complete elimination. Any difference in patient response could be due to the initial amounts of virus, to its genotype or to the specific immune response of the patient.

Details of the study are illustrated below.

### EXAMPLE

### Clinical Study

Ten female patients tested positive for HPV and subsequently confirmed as HPV-infected without immunologic diseases, were treated with low dosages of human natural interferon-α administered through the peroral route. A water solution was prepared dissolving 150 IU/ml human natural interferon-α in saline. The solution was stabilized with albumin and divided in 1 ml vials. For the treatment, one vial a day was administered for a period of 90 or, where necessary, 180 days.

At days 0, 30, 60, 90, 180 and 360 of treatment, a tissue-sample of uterus cervix was taken from each patient using an HC Cervical Sampler, and analysed with Hybrid Capture II kit and with 2HPV and CT/GC DNA tests (Diogene Corporation, USA).

Shortly, the Hybrid Capture II (HCII) test [Venturoli et al., J. Clin. Virol. 2002] is a liquid-phase hybridization assay utilizing RNA probes that discriminate 5 low-risk HPVs (6, 11, 42, 43 and 44). The DNA/RNA hybrid is immobilized on a plate by means of antibodies against double stranded DNA and detected by chemiluminescent-signal amplification.

The HCII kit was used for the detection of HPV in the lesion sites, whereas dosing and semi-quantitative determination of viral DNA copies in the sample (referred to 100000 cells) were performed with a PCR-ELISA test (J. Clin. Pathol.: 1998; 143-148, as modified in J. Clin. Pathol.: 2001; 54:377-380).

In short, PCR-ELISA was carried out with a consensus primer (MY11-MY09) able to amplify 30 low- and high-risk HPV genotypes. The amplification products were labeled with digoxigenin during the amplification reaction, separately hybridized to biotinylated probes specific for 7 low-risk (HPV 6, 11, 40, 42, 53, 54, 57) and 11 high-risk HPVs (16, 18, 31, 33, 35, 39, 45, 51, 52, 58, 59), immobilized on streptavidin-derivatized microplates and detected with immunoenzymatic assay (ELISA). A portion of the amplification product was analyzed electrophoretically to detect the amplified HPV which had not been typized with the available probes. Beta-globin was used as PCR-ELISA amplification control.

The assay provides a semiquantitative determination of the viral DNA copies in the sample based on the initial number of cells contained in the cervix sample and using calibration curves for each viral genotype.

The results are reported in the following table:

| | | HPV Day | HPV Day | HPV Day | HPV Day | HPV Day | HPV Day |
|---|---|---|---|---|---|---|---|
| Paz. | | 0 | 30 | 60 | 90 | 180 | 360 |
| n° | HPV-type | Index value | Index value | Index value | Index value | Index value | Index value |
| 1 | 6 | 11.000 | 5.300 | 350 | — | — | — |
| 2 | 53 | 70 | 60 | — | — | — | — |
| 3 | 11 | 250 | 200 | 50 | — | — | — |
| 4 | 6 | 120.000 | 52.500 | 400 | 90 | — | — |
| 5 | 6 | 20.000 | 13.000 | 150 | 50 | — | — |
| 6 | 42 | 450 | 250 | — | — | — | — |
| 7 | 11 | 250.000 | 110.000 | 800 | 60 | — | — |
| 8 | 6 | 2.500 | 1.650 | 180 | — | — | — |
| 9 | 11 | 25.000 | 12.000 | 250 | — | — | — |
| 10 | 54 | 900 | 600 | 100 | — | — | — |

The Table data show that in 7 out of 10 patients, namely patients 1, 2, 3, 6, 8, 9, 10, at day 90 (end of the treatment) the viral load was undetectable, while 3 patients out of 10, namely patients 4, 5, 7, showed a significantly reduced viral load. Patients of the second group were treated for additional 90 days. At day 180 and 360 of follow-up control, all patients resulted HPV-negative.

1-8. (canceled)

9. A method for the treatment of HPV infections, comprising administering to a subject in need thereof an effective amount of human interferon in a liquid composition.

10. The method according to claim 9, wherein the interferon is an amount of 100 to 500 IU/ml.

11. The method according to claim 10, wherein the interferon is in an amount of 150 IU/ml.

12. The method according to claim 9, wherein the human interferon is recombinant human interferon.

13. The method according to claim 12, wherein the interferon is natural interferon.

14. The method according to claim 9, wherein said liquid pharmaceutical composition is a water solution.

15. A method for the treatment of infections of the general tract, comprising administering to a subject in need thereof an effective amount of human interferon in a liquid composition.

16. A method for the treatment of warts or condylomatous lesions of the genital-tract mucosa, comprising administering to a subject in need thereof an effective amount of human interferon in a liquid composition.

17. The method according to claim 9, wherein the composition is administered properly.

18. The method according to claim 15, wherein the interferon is an amount of 100 to 500 IU/ml.

19. The method according to claim 15, wherein the interferon is in an amount of 150 IU/ml.

20. The method according to claim 15, wherein the human interferon is recombinant human interferon.

21. The method according to claim 15, wherein the interferon is natural interferon.

22. The method according to claim 15, wherein said liquid pharmaceutical composition is a water solution.

23. The method according to claim 16, wherein the interferon is an amount of 100 to 500 IU/ml.

24. The method according to claim 16, wherein the interferon is in an amount of 150 IU/ml.

25. The method according to claim 16, wherein the human interferon is recombinant human interferon.

26. The method according to claim 16, wherein the interferon is natural interferon.

27. The method according to claim 16, wherein said liquid pharmaceutical composition is a water solution.

28. The method according to claim 16, wherein the composition is administered properly.
